(19) United States
(12) Patent Application Publication
(10) Pub. No.: US 2013/0165530 A1
(43) Pub. Date: Jun. 27, 2013

(54) FOAMABLE ALCOHOLIC COMPOSITIONS WITH SKIN BENEFITS

(71) Applicant: GOJO INDUSTRIES, INC., Akron, OH (US)

(72) Inventors: Evan D. Hillman, North Canton, OH (US); C. Shawn Murphy, Harrison, OH (US)

(73) Assignee: GOJO INDUSTRIES, INC., Akron, OH (US)

(21) Appl. No.: 13/721,795

(22) Filed: Dec. 20, 2012

Related U.S. Application Data
(60) Provisional application No. 61/579,929, filed on Dec. 23, 2011.

(51) Int. Cl. A61K 8/04; A61Q 19/00

(52) U.S. Cl. CPC A61K 8/046; A61Q 19/00
USPC 514/738; 514/772.1

(57) ABSTRACT

A non-aqueous, non-aerosol foamable composition is provided that includes at least 40 wt % alcohol, based upon the total weight of the alcoholic composition; a foaming surfactant; and a skin benefit agent selected from the group consisting of emollients, humectants, occlusive agents, and other moisturizers to provide moisturization, skin softening, skin barrier maintenance, anti-irritation, or other skin health benefits. The foamable alcoholic composition forms a stable foam when mixed with air.

### TECHNICAL FIELD

This invention relates to foamable alcoholic compositions, and more particularly, to non-aqueous alcoholic compositions that may be foamed using non-aerosol dispensers to produce creamy, stable foam that is mild to the skin.

### BACKGROUND OF THE INVENTION

Foam cleaning products are popular, in part because they are easier to spread on surfaces. Consumers seem to prefer the luxury of foamed soap products. Less foam is needed to produce the same cleaning power as liquids or gels, due at least partly to the higher surface area of the foam. Properly formulated foam products do not produce the drip and splash that is experienced with traditional gelled or liquid products, due to the higher surface tension of the foam. This prevents damage to the floors and walls of facilities where the product dispensers are used. Manufacturing of foam products may be easier than gelled products, which often incorporate powdered thickeners that are difficult to handle.

Sanitizers for the skin should be skin-friendly. This is particularly true in situations where frequent use of the product is required, such as in health care settings and areas where food is prepared and served.

### SUMMARY OF THE INVENTION

One or more embodiments of this invention provide a non-aqueous, non-aerosol foamable composition comprising at least 40 wt % alcohol, based upon the total weight of the alcoholic composition; from about 0.02 to about 4 wt. % of a foaming surfactant; from about 0.1 to about 50 wt. % of a complex of hydrogen peroxide and a polymer formed from at least one vinyl heterocyclic monomer; and a skin benefit agent selected from the group consisting of emollients, humectants, occlusive agents, and other moisturizer to provide moisturization, skin softening, skin barrier maintenance, anti-irritation, or other skin health benefits, wherein the composition comprises from 0 to about 1 wt. % water, based upon the total weight of the composition.

One or more embodiments of the invention provide a non-aqueous, non-aerosol foamable composition comprising at least 40 wt % alcohol, based upon the total weight of the alcoholic composition; from about 0.02 to about 4 wt. % of a foaming surfactant; from about 0.1 to about 50 wt. % of a complex of hydrogen peroxide and a polymer formed from at least one vinyl heterocyclic monomer; and a skin benefit agent selected from the group consisting of emollients, humectants, occlusive agents, and other moisturizer to provide moisturization, skin softening, skin barrier maintenance, anti-irritation, or other skin health benefits, wherein the composition comprises from 0 to about 1 wt. % water, based upon the total weight of the composition, and wherein the composition, upon being mixed with air, forms a foam that is stable for at least about 3 minutes at room temperature.

One or more embodiments of this invention provide a method for forming a stabilized alcoholic foam, the method comprising the steps of combining a C₁₋₆ alcohol, a foaming surfactant, a non-aqueous skin benefit agent, a complex of hydrogen peroxide and a polymer formed from at least one vinyl heterocyclic monomer, and zero or less than 0.5 wt. % of added water, to form a foamable alcoholic composition; mixing said alcoholic composition and air or an inert gas in a mixing chamber to form a mixture; and passing said mixture through a mesh screen to form a foam, wherein said foamable alcoholic composition comprises at least about 40 percent by weight alcohol, based upon the total weight of the alcoholic composition, and wherein the the foam stability of the stabilized foam at room temperature is at least about 30 seconds.

### DETAILED DESCRIPTION OF ILLUSTRATIVE EMBODIMENTS

Foamable alcoholic compositions in accordance with this invention include at least one alcohol, at least one foaming surfactant, and at least one skin benefit agent. In one embodiment, the alcohol is a lower alkanol, i.e. an alcohol containing 1 to 6 carbon atoms. Typically, these alcohols have antimicrobial properties. Examples of lower alkanols include, but are not limited to, methanol, ethanol, propanol, butanol, pentanol, hexanol, and isomers and mixtures thereof. In one embodiment, the alcohol comprises ethanol, propanol, or butanol, or isomers or mixtures thereof. In another embodiment, the alcohol comprises ethanol.

Generally, the alcoholic composition comprises an amount of alcohol of at least about 20 weight percent (wt. %), based upon the total weight of the alcoholic composition. In one embodiment, the alcoholic composition comprises at least about 25 weight percent alcohol, in another embodiment, the alcoholic composition comprises at least about 30 weight percent alcohol, and in yet another embodiment, the alcoholic composition comprises at least about 35 weight percent alcohol, based upon the total weight of alcoholic composition. In one embodiment, the alcoholic composition comprises at least about 40 weight percent alcohol, in another embodiment, the alcoholic composition comprises at least about 45 weight percent alcohol, and in yet another embodiment, the alcoholic composition comprises at least about 50 weight percent alcohol, based upon the total weight of alcoholic composition. In one embodiment, the alcoholic composition comprises at least about 55 weight percent alcohol, in another embodiment, the alcoholic composition comprises at least about 60 weight percent alcohol, and in yet another embodiment, the alcoholic composition comprises at least about 65 weight percent alcohol, based upon the total weight of alcoholic composition. In one embodiment, the alcoholic composition comprises at least about 70 weight percent alcohol, in another embodiment, the alcoholic composition comprises at least about 75 weight percent alcohol, and in yet another embodiment, the alcoholic composition comprises at least about 80 weight percent alcohol, based upon the total weight of alcoholic composition. More or less alcohol may be required in certain instances, depending particularly on other ingredients and/or the amounts thereof employed in the composition. In certain embodiments, the alcoholic composition comprises from about 40 weight percent to about 98 weight percent alcohol, in other embodiments, the alcoholic composition comprises from about 45 weight percent to about 95 weight percent of alcohol, in yet other embodiments, the alcoholic composition comprises from about 50 weight percent to about 90 weight percent of alcohol, and in still other embodiments, the alcoholic composition comprises from about 60 weight percent to about 85 weight percent of alcohol, based upon the total weight of the alcoholic composition.

The foaming surfactant contributes foaming properties to the alcoholic composition, and may include anionic, cationic, nonionic, zwitterionic, or amphoteric surfactants and their associated salts. In one embodiment, the foaming surfactant includes a fluorosurfactant, a siloxane polymer surfactant, or a combination thereof Fluorosurfactants include compounds that contain at least one fluorine atom. Examples of fluorosurfactants include perfluoroalkylethyl phosphates, perfluoroalkylethyl betaines, fluoroaliphatic amine oxides, fluoroaliphatic sodium sulfosuccinates, fluoroaliphatic stearate esters, fluoroaliphatic phosphate esters, fluoroaliphatic quaternaries, fluoroaliphatic polyoxyethylenes, and the like, and mixtures thereof.

In one embodiment, the fluorosurfactant contains a charged species, i.e. is anionic, cationic, or zwitterionic. Examples of fluorosurfactants containing a charged species include perfluoroalkylethyl phosphates, perfluoroalkylethyl betaines, fluoroaliphatic amine oxides, fluoroaliphatic sodium sulfosuccinates, fluoroaliphatic phosphate esters, and fluoroaliphatic quaternaries. Specific examples of fluorosurfactants include DEA-C8-18 perfluoroalkylethyl phosphate, TEA-C8-18 perfluoroalkylethyl phosphate, NH₄-C8-18 perfluoroalkylethyl phosphate, and C8-18 perfluoroalkylethyl betaine.

Siloxane polymer surfactants may be generally characterized by containing one or more Si—O—Si linkages in the polymer backbone. The siloxane polymer surfactant may or may not include a fluorine atom. Therefore, some foaming surfactants may be classified as both fluorosurfactants and siloxane polymer surfactants. Siloxane polymer surfactants include organopolysiloxane dimethicone polyols, silicone carbinol fluids, silicone polyethers, alkylmethyl siloxanes, amodimethicones, trisiloxane ethoxylates, dimethiconols, quaternized silicone surfactants, polysilicones, silicone crosspolymers, and silicone waxes.

Examples of siloxane polymer surfactants include dimethicone PEG-7 undecylenate, PEG-10 dimethicone, PEG-8 dimethicone, PEG-12 dimethicone, perfluorononylethyl carboxydecal PEG 10, PEG-20/PPG-23 dimethicone, PEG-11 methyl ether dimethicone, bis-PEG/PPG-20/20 dimethicone, silicone quats, PEG-9 dimethicone, PPG-12 dimethicone, fluoro PEG-8 dimethicone, PEG 23/PPG 6 dimethicone, PEG 20/PPG 23 dimethicone, PEG 17 dimethicone, PEG5/PPG3 methicone, bis PEG20 dimethicone, PEG/PPG20/15 dimethicone copolyol and sulfosuccinate blends, PEG-8 dimethicone\dimmer acid blends, PEG-8 dimethicone\fatty acid blends, PEG-8 dimethicone\cold pressed vegetable oil\polyquaternium blends, random block polymers and mixtures thereof.

In one embodiment, the siloxane polymer surfactant includes a compound that may be represented by the formula

R²—Si (CH₃)₂—[O—Si (CH₃)₂]ₐ—[O—Si (CH₃) R³]_{b}—O—Si (CH₃)₂—R²

where R² and R³ independently include a methyl group or a moiety that may be represented by the formula

—(CH₂)₃—O—(CH₂CH₂O)_{c}—[CH₂CH(CH₃O]_{d}—(CH₂CH₂O)ₑ H

with the proviso that both R² and R³ are not CH₃ , where a is an integer from about 3 to about 21, b is an integer from about 1 to about 7, c is an integer from about 0 to about 40, d is an integer from about 0 to about 40, and e is an integer from about 0 to about 40, with the proviso that a ≧3×b and that c+d+e≧5.

The amount of foaming surfactant is not particularly limited, so long as an effective amount to produce foaming is present. In certain embodiments, the effective amount to produce foaming may vary, depending upon the amount of alcohol and other ingredients that are present. In one or more embodiments, the alcoholic composition includes at least about 0.002 wt. % of foaming surfactant, based upon the total weight of the alcoholic composition. In another embodiment, the alcoholic composition includes at least about 0.01 wt. % of foaming surfactant, based upon the total weight of the alcoholic composition. In yet another embodiment, the alcoholic composition includes at least about 0.05 wt. % of foaming surfactant, based upon the total weight of the alcoholic composition.

In one embodiment, the foaming surfactant is present in an amount of from about 0.002 to about 4 weight percent, based upon the total weight of the alcoholic composition. In another embodiment, the foaming surfactant is present in an amount of from about 0.01 to about 2 weight percent, based upon the total weight of the alcoholic composition. It is envisioned that higher amounts may also be effective to produce foam. All such weights as they pertain to listed ingredients are based on the active level and, therefore, do not include carriers or by-products that may be included in commercially available materials, unless otherwise specified.

In some embodiments, for economic or other reasons it may be desirable to limit the amount of fluorosurfactant used. Advantageously, stable foam can be produced from a composition according to the present invention containing greater than about 60 wt. % alcohol, and from about 0.002 to about 0.5 wt. % fluorosurfactant, based upon the total weight of the composition. In certain embodiments, the foamable composition includes greater than about 65 wt. % alcohol, and from about 0.002 to about 0.4 wt. % fluorosurfactant, based upon the total weight of the composition.

In other embodiments, it may be desirable to use higher amounts of foaming surfactant. For example, in certain embodiments where the foaming alcoholic composition of the present invention includes a cleansing or sanitizing product that is applied to a surface and then rinsed off, higher amounts of foaming surfactant may be employed. In these embodiments, the amount of foaming surfactant is present in amounts up to about 35 wt. %, based upon the total weight of the composition.

In one or more embodiments, the foaming surfactant is added directly to the alcoholic composition. In other embodiments, the foaming surfactant is added to the alcoholic composition as a solution or emulsion. In other words, the foaming surfactant may be premixed with a carrier to form a foaming surfactant solution or emulsion, with the proviso that the carrier is non-aqueous and does not deleteriously affect the foaming properties of the alcoholic composition. Examples of carriers include alcohol, glycols such as propylene or ethylene glycol, ketones, linear and/or cyclic hydrocarbons, triglycerides, carbonates, silicones, alkenes, esters such as acetates, benzoates, fatty esters, glyceryl esters, ethers, amides, polyethylene glycols and PEG/PPG copolymers, inorganic salt solutions such as saline, and mixtures thereof. It will be understood that, when the foaming surfactant is premixed to form a foaming surfactant solution or emulsion, the amount of solution or emulsion that is added to the alcoholic composition may be selected so that the amount of foaming surfactant falls within the ranges set forth hereinabove.

Foaming surfactant solutions and emulsions are commercially available. Some examples of commercially available dimethicone surfactants include Silsoft 810 and Silsoft 870 from Momentive Performance Materials, and Silsurf DI-1010 from Siltech.

In one or more embodiments, the foamable composition includes one or more skin benefit agents, such as an emollient, humectant, occlusive agent, or other moisturizer to provide moisturization, skin softening, skin barrier maintenance, anti-irritation, or other skin health benefits. Examples of skin benefit agents include oils, esters, silicones, glycols, glycerols, and ethers. In one or more embodiments, skin benefit agents include propylene glycol and ethylene glycol.

Advantageously, the alcoholic composition of these embodiments produce a stable foam when mixed with air, and do not require a foam stabilizer. For purposes of this specification, a stable foam is one that maintains a measurable height for at least about 5 seconds following creation of the foam. Thus, by “foam stability” is meant the length of time that it takes for a foam to break down into a liquid.

Optionally, one or more polymeric foam stabilizers may be employed. Thus, in certain embodiments, the alcoholic composition of the present invention further includes at least one foam stabilizer. The foam stabilizer may be polymeric or non-polymeric. In one embodiment, the foam stabilizer may be selected from polymeric or oligomeric foam stabilizers. In one embodiment, the foam stabilizer comprises a cationic oligomer or polymer.

Polymeric foam stabilizers include polyquaternium polymers. In general, a polyquaternium polymer is one that is designated as such by the CTFA. Polyquaternium polymers may be characterized by containing a quaternary ammonium group. Polyquaternium foam stabilizers are further described in U.S. Patent Application Pub. No. 2007/0148101 A1, which is hereby incorporated by reference herein.

Other foam stabilizers that may operate to improve foam quality and/or stability include terpolymers of vinylcaprolactam (VCL), vinylpyrrolidone (VP) and dialkylaminoalkyl acrylate, including a VP/vinylcaprolactam/dimethylaminopropyl methacrylamide copolymer. Yet another foam stabilizer includes isobutylene/dimethylaminopropyl maleimide/ethoxylated maleimide/maleic acid copolymer. These and other foam stabilizers are sometimes referred to as film-forming polymers.

Still other foam stabilizers include acrylamide/ammonium acrylate copolymer, acrylamides/DMAPA acrylates/methoxy PEG methacrylate copolymer, acrylamide/sodium acryloyldimethyltaurate/acrylic acid copolymer, acrylamidopropyltrimonium chloride/acrylamide copolymer, acrylamidopropyltrimonium chloride/acrylates copolymer, acrylates/acetoacetoxyethyl methacrylate copolymer, acrylates/acrylamide copolymer, acrylates/ammonium methacrylate copolymer, acrylates/t-butylacrylamide copolymer, acrylates copolymer, acrylates/C1-2 succinates/hydroxyacrylates copolymer, acrylates/ethylamine oxide methacrylate copolymer, acrylates/lauryl acrylate/stearyl acrylate/ethylamine oxide methacrylate copolymer, acrylates/octylacrylamide copolymer, acrylates/octylacrylamide/diphenyl amodimethicone copolymer, acrylates/polytrimethyl siloxymethacrylate copolymer, acrylates/stearyl acrylate/ethylamine oxide methacrylate copolymer, acrylates/trifluoropropylmethacrylate/polytrimethyl siloxymethacrylate copolymer, acrylates/VA copolymer, acrylates/VP copolymer, adipic acid/diethylenetriamine copolymer, adipic acid/dimethylaminohydroxypropyl diethylenetriamine copolymer, adipic acid/epoxypropyl diethylenetriamine copolymer, adipic acid/isophthalic acid/neopentyl glycol/trimethylolpropane copolymer, allyl stearate/VA copolymer, aminoethylacrylate phosphate/acrylates copolymer, aminoethylpropanediol-acrylates/acrylamide copolymer, aminoethylpropanediol-AMPD-acrylates/diacetoneacrylamide copolymer, ammonium VA/acrylates copolymer, amodimethicone/silsesquioxane copolymer, AMPD-acrylates/diacetoneacrylamide copolymer, AMP-acrylates/allyl methacrylate copolymer, AMP-acrylates/C1-18 alkyl acrylates/C1-8 alkyl acrylamide copolymer, AMP-acrylates/diacetoneacrylamide copolymer, AMP-acrylates/dimethylaminoethylmethacrylate copolymer, bacillus/rice bran extract/soybean extract ferment filtrate, behenyl methacrylate/ethylamine oxide methacrylate copolymer, bis-butyloxyamodimethicone/PEG-60 copolymer, bis-isobutyl PEG-14/amodimethicone copolymer, bis-isobutyl PEG-15/amodimethicone copolymer, butyl acrylate/ethylhexyl methacrylate copolymer, butyl acrylate/hydroxypropyl dimethicone acrylate copolymer, butyl ester of ethylene/MA copolymer, butyl ester of PVM/MA copolymer, calcium/sodium PVM/MA copolymer, chitosan, chitosan lactate, corn starch/acrylamide/sodium acrylate copolymer, dehydroxanthan gum, diethylene glycolamine/epichlorohydrin/piperazine copolymer, dimethicone crosspolymer, dimethicone/silsesquioxane copolymer, diphenyl amodimethicone, ethyl ester of PVM/MA copolymer, ethyltrimonium chloride methacrylate/hydroxyethylacrylamide copolymer, hydrolyzed wheat protein/PVP crosspolymer, hydroxypropyl dimethiconylpropyl acrylates copolymer, hydroxypropyltrimonium hydrolyzed corn starch, isobutylene/ethylmaleimide/hydroxyethylmaleimide copolymer, isobutylene/MA copolymer, isobutylmethacrylate/trifluoroethylmethacrylate/bis-hydroxypropyl dimethicone acrylate copolymer, isopropyl ester of PVM/MA copolymer, lauryl acrylate crosspolymer, lauryl methacrylate/glycol dimethacrylate crosspolymer, lauryl PEG-9 polydimethylsiloxyethyl dimethicone, methacrylic acid/sodium acrylamidomethyl propane sulfonate copolymer, methacryloyl ethyl betaine/acrylates copolymer, methoxy amodimethicone/silsesquioxane copolymer, methoxy PEG-114/polyepsilon caprolactone, myristic/palmitic/stearic/ricinoleic/eicosanedioic glycerides, octylacrylamide/acrylates/butylaminoethyl methacrylate copolymer, PEG-800/polyvinyl alcohol copolymer, PEG/PPG-25/25 dimethicone/acrylates copolymer, PEG-8/SMDI copolymer, polyacrylamide, polyacrylate-6, polyacrylate-8, polyacrylate-9, polyacrylate-15, polyacrylate-16, polyacrylate-17, polyacrylate-18, polyacrylate-19, polybeta-alanine/glutaric acid crosspolymer, polybutylene terephthalate, polyester-1, polyethylacrylate, polyethylene terephthalate, polyimide-1, polymethacryloyl ethyl betaine, polypentaerythrityl terephthalate, polyperfluoroperhydrophenanthrene, polyquaternium-4/hydroxypropyl starch copolymer, polyurethane-1, polyurethane-6, polyurethane-10, polyurethane-18, polyurethane-19, polyvinyl acetate, polyvinyl butyral, polyvinylcaprolactam, polyvinylformamide, polyvinyl imidazolinium acetate, polyvinyl methyl ether, potassium butyl ester of PVM/MA copolymer, potassium ethyl ester of PVM/MA copolymer, PPG-70 polyglyceryl-10 ether, PPG-12/SMDI copolymer, PPG-51/SMDI copolymer, PVM/MA copolymer, PVP/VA/itaconic acid copolymer, PVP/VA/vinyl propionate copolymer, rhizobian gum, rosin acrylate, shellac, silicone quaternium-16/glycidoxy dimethicone crosspolymer, sodium butyl ester of PVM/MA copolymer, sodium ethyl ester of PVM/MA copolymer, sodium polyacrylate, sodium polygamma-glutamate, soy protein phthalate, sterculia urens gum, terephthalic acid/Isophthalic acid/sodium isophthalic acid sulfonate/glycol copolymer, trimethylolpropane triacrylate, trimethylsiloxysilylcarbamoyl pullulan, VA/crotonates copolymer, VA/crotonates/methacryloxybenzophenone-1 copolymer, VA/crotonates/vinyl neodecanoate copolymer, VA/crotonates/vinyl propionate copolymer, VA/DBM copolymer, VA/vinyl butyl benzoate/crotonates copolymer, vinylamine/vinyl alcohol copolymer, vinyl caprolactam/VP/dimethylaminoethyl methacrylate copolymer, VP/acrylates/lauryl methacrylate copolymer, VP/dimethylaminoethylmethacrylate copolymer, VP/DMAPA acrylates copolymer, VP/hexadecene copolymer, VP/methacrylamide/vinyl imidazole copolymer, VP/VA copolymer, VP/vinyl caprolactam/DMAPA acrylates copolymer, yeast palmitate, a silicon-based polymer or resin such as phenylpropyldimethyl siloxysilicate, trimethylsiloxysilic ate, cyclopentasiloxane, trimethylsiloxysilicate, diisostearoyl trimethyllolpropane siloxy silicate, vinyl dimethicone crosspoylmer/blends, and alkyl cetearyl dimethicone crosspolymers.

Commercially available foam stabilizers include a VP/vinylcaprolactam/dimethyl-aminopropyl methacrylamide copolymer sold under the trade name Aquaflex SF-40, and an isobutylene/dimethylaminopropyl maleimide/ethoxylated maleimide/maleic acid copolymer sold under the trade name Aquaflex XL-30.

In one embodiment, foam stabilizer is present in an amount of from about 0.005 to about 4 weight percent active, based upon the total weight of the alcoholic composition. In another embodiment, the foam stabilizer is present in an amount of from about 0.01 to about 1 weight percent, based upon the total weight of the alcoholic composition, and in yet another embodiment, the foam stabilizer is present in an amount of from about 0.02 to about 0.2 weight percent, based upon the total weight of the alcoholic composition.

In one embodiment, the foam stabilizer is added directly to the alcoholic composition. In one or more other embodiments, the foam stabilizer is added to the alcoholic composition as a solution or emulsion. In other words, the foam stabilizer may be premixed with a carrier to form a foam stabilizer solution or emulsion, with the proviso that the carrier in non-aqueous and does not deleteriously affect the foaming properties of the alcoholic composition. Examples of carriers include alcohol, glycols such as propylene or ethylene glycol, ketones, linear and/or cyclic hydrocarbons, triglycerides, carbonates, silicones, alkenes, esters such as acetates, benzoates, fatty esters, glyceryl esters, ethers, amides, polyethylene glycols and PEG/PPG copolymers, inorganic salt solutions such as saline, and mixtures thereof. It will be understood that, when the foam stabilizer is premixed to form a foam stabilizer solution or emulsion, the amount of solution or emulsion that is added to the alcoholic composition is selected so that the amount of foam stabilizer falls within the ranges set forth hereinabove.

In one embodiment, the weight ratio of foaming surfactant to foam stabilizer is from about 0.5:1 to about 75:1, in another embodiment from about 3.5:1 to about 45:1, and in another embodiment, the weight ratio of foaming surfactant to foam stabilizer is from about 8:1 to about 15:1.

In one embodiment, the foam stability of the foam produced when the foamable alcoholic composition of the present invention is passed through a non-aerosol foaming pump at room temperature is at least about 30 seconds, in another embodiment at least about three minutes, or in other words, the alcoholic composition maintains its foam form and doesn't break down into liquid form for at least three minutes. In another embodiment, the foam stability is at least about five minutes, and in yet another embodiment, the foam stability is at least about 15 minutes. In one or more embodiments, the foam stability is at least about 30 minutes, and in other embodiments, the foam stability is at least about 60 minutes.

The alcoholic composition of this invention may further include a wide range of optional ingredients, with the proviso that they are substantially non-aqueous and do not deleteriously affect the foam forming properties of the alcoholic composition, or the stability of the foam. The CTFA International Cosmetic Ingredient Dictionary and Handbook, Eleventh Edition, 2005, and the 2004 CTFA International Buyer's Guide, both of which are incorporated by reference herein in their entirety, describe a wide variety of non-limiting cosmetic and pharmaceutical ingredients commonly used in the skin care industry, that are suitable for use in the compositions of the present invention. Non-limiting examples of functional classes of ingredients are described in these references. Examples of these functional classes include: abrasives, anti-acne agents, anticaking agents, antioxidants, binders, biological additives, bulking agents, chelating agents, chemical additives; colorants, cosmetic astringents, cosmetic biocides, denaturants, drug astringents, emulsifiers, external analgesics, film formers, fragrance components, humectants, opacifying agents, plasticizers, preservatives, propellants, reducing agents, skin bleaching agents, skin-conditioning agents (emollient, humectants, miscellaneous, and occlusive), skin protectants, solvents, foam boosters, hydrotropes, solubilizing agents, suspending agents (nonsurfactant), sunscreen agents, ultraviolet light absorbers, detackifiers, and viscosity increasing agents (aqueous and nonaqueous). Examples of other functional classes of materials useful herein that are well known to one of ordinary skill in the art include solubilizing agents, sequestrants, and keratolytics, and the like. In one or more embodiments, the alcoholic composition further comprises from about 0.005 to about 10 wt. % of glycerin. In one or more embodiments, the alcoholic composition further comprises from about 0.005 to about 4 wt. % of at least one 1,2-alkane diol. In one or more embodiments, the alcoholic composition further comprises from about 0.005 to about 4 wt. % of 1,2-octane diol. In one or more embodiments, the alcoholic composition further comprises from about 0.1 to about 50 wt. % hydrogen peroxide, or of a hydrogen peroxide/polymer complex. In one or more embodiments, the alcoholic composition further comprises from about 0.005 to about 10 wt. % of glycerin, from about 0.005 to about 4 wt. % of at least one 1,2-alkane diol, and from about 0.1 to about 50 wt. % hydrogen peroxide, or of a hydrogen peroxide/polymer complex.

Auxiliary surfactants may be included in the alcoholic compositions for the purpose of boosting or modifying the foam quality and characteristics, for modifying the feel of the final formulation during rub in and/or dry time, for providing persistence or long-lasting microbial action of the alcohol, for solubilizing other ingredients such as fragrances or sunscreens, and for irritation mitigation. Auxiliary surfactants include, but are not necessarily limited to, sulfosuccinates, amine oxides, PEG-80 sorbitan laurate, polyglucosides, alkanolamides, sorbitan derivatives, fatty alcohol ethoxylates, quaternary ammonium compounds, amidoamines, sultaines, isothionates, sarcosinates, betaines, and fatty alcohol polyethylene glycols.

Although a propellant may be used to produce stable foam, advantageously a propellant is not necessary. In certain embodiments, the amount of propellant is less than about 1000 parts per million by weight, based upon the total weight of the alcoholic composition. In one embodiment, the alcoholic composition is substantially free of propellants, such as hydrocarbon propellants. By substantially free is meant that the amount of propellant in the alcoholic composition is less than about 100 parts per million by weight, based upon the total weight of the alcoholic composition.

In one embodiment, alcohol is the only active antimicrobial ingredient introduced into the composition, and in this embodiment the amount of auxiliary antimicrobial ingredients is less than about 0.1 weight percent, based upon the total weight of the alcoholic composition.

In other embodiments, the composition includes auxiliary antimicrobial agents in addition to alcohol. Examples of auxiliary antimicrobial agents include, but are not limited to, triclosan, also known as 5-chloro-2(2,4-dichlorophenoxy) phenol and available from Ciba-Geigy Corporation under the tradename IRGASAN®; chloroxylenol, also known as 4-chloro-3,5-xylenol, available from Nipa Laboratories, Inc. under the tradenames NIPACIDE® MX or PX; hexetidine, also known as 5-amino-1,3-bis(2-ethylhexyl)-5-methyl-hexahydropyrimidine; chlorhexidine salts including chlorhexidine gluconate and the salts of N,N″ -Bis(4-chlorophenyl)-3,12-diimino-2,4,11,14-tetraazatetradecanediimidi amide; 2-bromo-2-nitropropane-1; 3-diol, benzalkonium chloride; cetylpyridinium chloride; alkylbenzyldimethylammonium chlorides; iodine; phenol derivatives, povidone-iodine including polyvinylpyrrolidinone-iodine;

parabens; hydantoins and derivatives thereof, including 2,4-imidazolidinedione and derivatives of 2,4-imidazolidinedione as well as dimethylol-5,5-dimethylhydantoin (also known as DMDM hydantoin or glydant); phenoxyethanol; cis isomer of 1-(3-chloroallyl)-3,5,6-triaza-1-azoniaadamantane chloride, also known as quaternium-15 and available from Dow Chemical Company under the tradename DOWCIL™ 2000; diazolidinyl urea; benzethonium chloride; methylbenzethonium chloride; and mixtures thereof. When used, the auxiliary antimicrobial agents are present in amounts of from about 0.1 to about 1 wt. %, based upon the total weight of the alcoholic composition.

In one or more embodiments, the foamable composition may include a complex of hydrogen peroxide and of a polymer comprising, as monomer, at least one vinyl heterocyclic monomer. In one or embodiments, the vinyl heterocyclic monomer is chosen from monomers comprising a 4- to 6-membered heterocycle, optionally fused to a benzene ring, which comprises from 1 to 4 identical or different intracyclic heteroatoms; the number of intracyclic heteroatoms being less than that of the ring members of the heterocycle. In one or more embodiments, the number of intracyclic heteroatoms is 1 or 2.

In one or more embodiments, the heteroatom or heteroatoms may be chosen from sulphur, oxygen or nitrogen, preferably from nitrogen and oxygen. In certain embodiments, the monomer comprises at least one intracyclic nitrogen atom.

The vinyl heterocycle can optionally be substituted by one or more C₁-C₄. In one or more embodiments, The vinyl heterocycle can optionally be substituted by one or more C₁-C₂, alkyl groups.

In one or more embodiments, the heterocyclic monomer is chosen from N-vinyl monomers. Examples of suitable monomers include substituted: N-vinylpyrrolidone, vinylcaprolactam, N-vinylpiperidone, N-vinyl-3-morpholine, N-vinyl-4-oxazolinone, 2-vinylpyridine, 4-vinyl-pyridine, 2-vinylquinoline, 1-vinylimidazole or 1-vinylcarbazole. In one or more embodiments, the monomer is optionally substituted N-vinylpyrrolidine.

In one or more embodiments, the polymer is a homopolymer. In other embodiments, the polymer is a copolymer. Examples of suitable comonomers include vinyl acetate, (meth)acrylic acids, (meth)acrylamides, or C₁-C₄ alkyl esters of (meth)acrylic acid, which may or may not be substituted. In one or more embodiments, the polymer is polyvinylpyrrolidone (PVP). In one or more embodiments, the hydrogen peroxide polymer complex is a product that is denoted by the International Nomenclature of Cosmetic Ingredients (INCI) as PVP (and) Hydrogen Peroxide.

The polymer involved in this complex can furthermore be soluble or insoluble in water. Preferably, it is soluble in water. It can exert variable average molecular weights, preferably between 10³ and 3×10⁶ g/mol. It is also possible to employ blends of such polymers.

In one or more embodiments, the complex comprises from 10 to 30 wt. % of hydrogen peroxide, more particularly from 13 to 25 wt. % and preferably from 18 to 22 wt. %, with respect to the total weight of the complex.

In one or more embodiments, the molar ratio of the vinyl heterocyclic monomer or monomers to the hydrogen peroxide in the complex ranges from 0.5 to 2, preferably from 0.5 to 1.

The complex may be provided in the form of a substantially anhydrous powder. In one or more embodiments, the powder comprises less than 5 wt. % of water, in other embodiments the powder comprises from zero to about 1 wt. % of water.

Examples of suitable complexes of polymer and peroxide are further described in U.S. Pat. No. 5,008,106 and U.S. Pat. No. 5,077,047, both of which are hereby incorporated by reference.

Examples of commercially available polymer-peroxide complex products include Peroxydone K-30, Peroxydone KS90, Peroxydone XL-10, and the like, and also the complexes formed with hydrogen peroxide and one of the following polymers of Plasdone K-17, Plasdone K-25, Plasdone K-29/32, Plasdone K-90, Polyplasdone INF-10, Polyplasdone

XL-10, Polyplasdone XL, Plasdone S-630, Styleze 2000 terpolymer or Ganex copolymers series type, available from ISP.

The non-aqueous foamable alcoholic compositions according to the invention advantageously comprises from about 0.1 to about 50 wt. % of the polymer-peroxide complex, in one or more embodiments, from about 0.5 to about 30 wt. %, in one or more embodiments from about 1 to about 25 wt. %, based upon the total weight of the composition.

The alcoholic composition of the present invention may optionally further comprise a wide range of topical drug actives, with the proviso that they do not deleteriously affect the foam forming properties of the alcoholic composition, or the stability of the foam. Examples of topical drug actives include salicylic acid, acetyl salicylic acid, cis-retinoic acid, trans-retinoic acid, N-acetyl-L-cysteine, lipoic acid, azelaic acid, phytic acid, lisophosphotidic acid, tetracycline, ibuprofen, naproxen, acetominophen, hydrocortisone, resorcinol, phenoxyethanol, phenoxypropanol, phenoxyisopropanol, 2,4,4′-trichloro-2′-hydroxy diphenyl ether, 3,4,4′-trichlorocarbanilide, octopirox, 2-phenylbenzimidazole-5-sulfonic acid, dihydroxyacetone, benzoyl peroxide, 2,4,4′-trichloro-2-hydroxy diphenyl ether, phenoxyethanol, phenoxypropanol, phenoxyisopropanol, phytic acid, lipoic acid, lisophosphatidic acid, benoxaprofen, flubiprofen, fenoprofen, fenbufen, ketoprofen, indoprofen, priprofen, carprofen, oxaprozin, pranoprofen, miroprofen, tioxaprofen, suprofen, alminoprofen, tiaprofenic acid, fluprofen, bucloxic acid, benzocaine, lidocaine, bupivacaine, chloroprocaine, dibucaine, etidocaine, mepivacaine, tetracaine, dyclonine, hexylcaine, procaine, cocaine, ketamine, pramoxine, phenol, dihydroxyacetone, tyrosine, ethyltryosinate, phospho-DOPA, β-lactim drugs, quinoline drugs, ciprofloxacin, norfloxacin, erythromycin, amikacin, doxycycline, capreomycin, chlorhexidine, chlortetracycline, oxytetracycline, clindamycin, ethambutol, hexamidinee isethionate, metronidazole, pentamidine, gentamicin, kanamycin, lineomycin, methacyclin, methenamine, minocycine, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycicyline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, amanfadine hydrochloride, amnanfadine sulfate, octopirox, parachlorometa xylenol, nystatin, tolnaftate, clotrimazole, 2-ethylhexyl p-methoxycinnamate, octyl methoxycinnamate, p-amino benzoate, p-aminobenzoic acid, 2-phenyl benzimidazole-5-sulfonic acid, octocrylene, oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, titanium dioxide, silica, iron oxide, 4-N,N-(2-ethylhexyl)methyl aminobenzoic acid ester of 2,4-dihydroxybenzophenone, 4-N,N-(2-ethylhexyl)methyl aminobenzoic acid ester with 4-hydroxydibenzoylmethane, 4-N,N-(2-ethylhexyl)methyl aminobenzoic acid ester of 2-hydroxy-4-(2-hydroxyethoxy)benzophenone, 4-N,N-(2-ethylhexyl)-methyl aminobenzoic acid ester of 4-(2-hydroxyethoxy)dibenzoylmethane, tetracycline, ibuprofen, naproxen, acetaminophen, resorcinol, 3,4,4′-trichlorocarbanilide, octopirox, pharmaceutically-acceptable salts and mixtures of the above.

In one or more embodiments, the balance of the alcoholic composition includes a non-aqueous solvent or other non-aqueous component. In one embodiment, the balance of the composition is a skin benefit agent such as propylene glycol or ethylene glycol. Foamable alcoholic compositions in accordanc3 with this invention are non-aqueous. That is, the amount of water is de minimus. In one or more embodiments, the foamable alcoholic composition comprises less than about 1 wt. % water, in other embodiments less than about 0.5 wt. %, in yet other embodiments, less than about 0.2 wt. %, in still other embodiments, less than about 0.1 wt. % water, based upon the total weight of the foamable alcoholic composition. In one or more embodiments, the foamable alcoholic composition is devoid of water. Surprisingly, water is not needed in order to produce a stable, non-aerosol alcohol foam.

Stated another way, in one or more embodiments, the composition comprises from 0 to about 1 wt. % water, based upon the total weight of the composition, in other embodiments from about 0 to about 0.5 wt. % water, in yet other embodiments, from about 0 to about 0.1 wt. % water, based upon the total weight of the composition.

The alcoholic composition may be prepared by simply mixing the components together. The order of addition is not particularly limited. In one or more embodiments, the components are combined and mixed to form a substantially homogeneous mixture. In one or more embodiments, the mixture is substantially clear.

The foamable composition of the present invention may be employed in any type of dispenser typically used for foam products. Advantageously, while the foamable composition can optionally be foamed by aerosolizing the composition, an aerosolized product is not necessary for foaming. Any dispenser that is capable of mixing the foamable alcoholic composition with air or an inert gas may be used. Inert gases include gas that does not substantially react or otherwise deleteriously affect the foamable composition. Examples of inert gases include nitrogen, argon, xenon, krypton, helium, neon, and radon. In one embodiment, the alcoholic composition is used in dispensers that employ foaming pumps, which combine ambient air or an inert gas and the alcoholic composition in a mixing chamber and pass the mixture through a mesh screen. In this and other embodiments, the viscosity of the composition is less than about 500 mPas, in one or more embodiments, less than about 100 mPas, in one or more embodiments less than about 50 mPas, and other embodiments less than about 25mPas, as measured by a Brookfield RV Viscometer using RV and/or LV Spindles at 22 oC +/−3 oC.

The alcoholic composition of the present invention produces stabilized foam. As a result, the foamed composition will be less likely to break down to a liquid state and drip from the foaming pump and/or associated dispenser elements.

Accordingly, the present invention further provides a method of reducing dripping of a composition from a foaming pump. Examples of foaming pumps include Airspray® foaming pumps. When a foamable composition is dispensed by using a foamable pump, a portion of the dispensed composition, now in foam form, remains in the dispenser head until another aliquot is dispensed. If the foam in the dispenser head breaks down into liquid between uses of the dispenser, the liquid will drip out of the dispenser head. The time between uses of the dispenser can be designated generally as X minutes. “X” can vary widely.

Advantageously, the stabilized foam of the present invention does not drip from the dispenser when X is about 3 minutes or less. In one embodiment, the stabilized foam does not drip from the dispenser when X is about five minutes or less, and in another embodiment, when X is about 15 minutes or less. In one or more embodiments, the stabilized foam does not drip from the dispenser when X is about 30 minutes or less, and in other embodiments, the stabilized foam does not drip from the dispenser when X is about 60 minutes or less.

Advantageously, the alcoholic composition of the present invention provides surprising skin benefits. This advantage is most evident in situations where the product must be used frequently.

In order to demonstrate the practice of the present invention, the following examples have been prepared and tested. The examples should not, however, be viewed as limiting the scope of the invention. The claims will serve to define the invention.

### EXAMPLES

### Example 1

### Foamable Alcoholic Composition

| | WEIGHT PERCENT |
|---|---|
| INGREDIENT | (WT. %) |
| Ethanol | 40-99 |
| Foaming surfactant (PEG-10 dimethicone*) | 0.02-4 |
| Skin benefit agent (ethylene glycol) | q.s. |

*DI 1010, available from Siltech Industries.

### Example 2

### Foamable Alcoholic Composition

| | WEIGHT PERCENT |
|---|---|
| INGREDIENT | (WT. %) |
| Ethanol | 40-99 |
| Foaming surfactant (PEG-10 dimethicone*) | 0.02-4 |
| Glycerin | 0.005-10 |
| 1,2-alkane diol (1,2-octane diol) | 0.005-4 |
| Skin benefit agent (ethylene glycol) | q.s. |

*DI 1010, available from Siltech Industries.

### Example 3

### Foamable Alcoholic Composition

| | WEIGHT PERCENT |
|---|---|
| INGREDIENT | (WT. %) |
| Ethanol | 40-99 |
| Foaming surfactant (PEG-10 dimethicone*) | 0.02-4 |
| H2O2-polymer (H2O2-PVP**) | 0.1-50 |
| Skin benefit agent (ethylene glycol) | q.s. |

*DI 1010, available from Siltech Industries.
**INCI name PVP (and) Hydrogen Peroxide, available from ISP under the tradename Peroxydone K-90.

### Example 4

### Foamable Alcoholic Composition

| | WEIGHT PERCENT |
|---|---|
| INGREDIENT | (WT. %) |
| Ethanol | 40-99 |
| Foaming surfactant (PEG-10 dimethicone*) | 0.02-4 |
| H2O2-polymer (H2O2-PVP**) | 0.1-50 |
| Glycerin | 0.005-10 |
| 1,2-alkane diol (1,2-octane diol) | 0.005-4 |
| Skin benefit agent (ethylene glycol) | q.s. |

*DI 1010, available from Siltech Industries.
**INCI name PVP (and) Hydrogen Peroxide, available from ISP under the tradename Peroxydone K-90.

Various modifications and alterations that do not depart from the scope and spirit of this invention will become apparent to those skilled in the art. This invention is not to be duly limited to the illustrative embodiments set forth herein.

What is claimed is:

1. A non-aqueous, non-aerosol foamable composition comprising:
at least 40 wt % alcohol, based upon the total weight of the alcoholic composition;
from about 0.02 to about 4 wt. % of a foaming surfactant;
from about 0.1 to about 50 wt. % of a complex of hydrogen peroxide and a polymer formed from at least one vinyl heterocyclic monomer; and
a skin benefit agent selected from the group consisting of emollients, humectants, occlusive agents, and other moisturizer to provide moisturization, skin softening, skin barrier maintenance, anti-irritation, or other skin health benefits,
wherein the composition comprises from 0 to about 1 wt. % water, based upon the total weight of the composition.

2. The composition of claim 1, wherein said skin benefit agent is selected from the group consisting of glycols, glycerols, oils, esters, silicones, and ethers.

3. The composition of claim 2, wherein said skin benefit agent is selected from the group consisting of propylene glycol and ethylene glycol.

4. The composition of claim 1, where said composition comprises less than about 0.5 wt. % water, based upon the total weight of the composition.

5. The composition of claim 4, where said composition comprises less than about 0.1 wt. % water, based upon the total weight of the composition.

6. The composition of claim 1, where said composition is devoid of water.

7. The composition of claim 1, where the foaming surfactant is a siloxane polymer surfactant.

8. The composition of claim 7, where the skin benefit agent is propylene glycol or ethylene glycol.

9. The composition of claim 8, where the complex of hydrogen peroxide and a polymer formed from at least one vinyl heterocyclic monomer is a complex of hydrogen peroxide and polyvinylpyrrolidone.

10. The composition of claim 7, wherein the composition further comprises one or more foam enhancers.

11. A non-aqueous, non-aerosol foamable composition comprising:
at least 40 wt % alcohol, based upon the total weight of the alcoholic composition;
from about 0.02 to about 4 wt. % of a foaming surfactant;
from about 0.1 to about 50 wt. % of a complex of hydrogen peroxide and a polymer formed from at least one vinyl heterocyclic monomer; and
a skin benefit agent selected from the group consisting of emollients, humectants, occlusive agents, and other moisturizer to provide moisturization, skin softening, skin barrier maintenance, anti-irritation, or other skin health benefits,
wherein the composition comprises from 0 to about 1 wt. % water, based upon the total weight of the composition, and wherein the composition, upon being mixed with air, forms a foam that is stable for at least about 3 minutes at room temperature.

12. A method for forming a stabilized alcoholic foam, the method comprising the steps of:
combining a C₁₋₆ alcohol, a foaming surfactant, a non-aqueous skin benefit agent, a complex of hydrogen peroxide and a polymer formed from at least one vinyl heterocyclic monomer, and zero or less than 0.5 wt. % of added water, to form a foamable alcoholic composition;
mixing said alcoholic composition and air or an inert gas in a mixing chamber to form a mixture; and
passing said mixture through a mesh screen to form a foam, wherein said foamable alcoholic composition comprises at least about 40 percent by weight alcohol, based upon the total weight of the alcoholic composition, and wherein the the foam stability of the stabilized foam at room temperature is at least about 30 seconds.

13. The method of claim 12, wherein the foamable complsition comprises at least 40 wt % alcohol, based upon the total weight of the alcoholic composition; from about 0.02 to about 4 wt. % of a foaming surfactant selected from the group consisting of anionic, cationic, and zwitterionic fluorosurfactants, and combinations thereof; from about 0.1 to about 50 wt. % of a complex of hydrogen peroxide and a polymer formed from at least one vinyl heterocyclic monomer; and a skin benefit agent selected from the group consisting of emollients, humectants, occlusive agents, and other moisturizer to provide moisturization, skin softening, skin barrier maintenance, anti-irritation, or other skin health benefits, wherein the composition comprises from 0 to about 1 wt. % water, based upon the total weight of the composition.
